(19) United States
(12) Patent Application Publication
(10) Pub. No.: US 2007/0191796 A1
(43) Pub. Date: Aug. 16, 2007

(54) Side panel for absorbent article

(75) Inventors: Michael Vincent, Toronto (CA); Stanley Littman, Roswell, GA (US); Mitchell Larson, Alpharetta, GA (US)

Correspondence Address: HAMRE, SCHUMANN, MUELLER & LARSON, P.C., P.O. BOX 2902, MINNEAPOLIS, MN, 55402-0902, US

(73) Assignee: IRVING PERSONAL CARE LIMITED, DIEPPE (CA)

(21) Appl. No.: 11/355,909

(22) Filed: Feb. 16, 2006

(51) Int. Cl. A61F 13/15

(52) U.S. Cl. 604/358; 428/221

(57) ABSTRACT

An elasticized retaining panel for an absorbent garment has a stretch axis and a cross axis normal to the stretch axis, the panel including an outer web, an inner web overlying the outer web, and an elastic web disposed between the outer and inner webs and bonded thereto. The elastic web includes a plurality of stretch strands extending parallel to the stretch axis and a plurality of support strands extending parallel to the cross axis. The support strands and stretch strands define a plurality of intersection points at which the support strands and the stretch strands are joined together. The stretch strands are elastically extendible along the stretch axis and are extended relative to the outer and inner webs parallel to the stretch axis for exerting an elastically contractile force on the outer and inner webs. The support strands are generally unextended relative to the inner and outer webs parallel to the cross axis.

### FIELD

This specification relates to absorbent articles, including, for example, diapers or training pants, and to elasticized side panel structures therefor.

### BACKGROUND

U.S. Pat. No. 4,938,757 (Van Gompel et al.) discloses a disposable pant-like garment for absorbing human discharge having an absorbent assembly including a liquid impervious outer cover, a liquid pervious liner, and an absorbent medium therebetween. A pair of stretchable side panels are joined to the absorbent assembly to form a waist opening and a pair of leg openings, and an intermediate portion of each stretchable side panel is generally peripherally disposed about a respective leg opening. The stretchable side panels provide generally inwardly directed force vectors against a wearer to maintain the garment snugly against the wearer's body and the absorbent assembly snugly in place against the crotch area both before and after a discharge.

U.S. Pat. No. 5,415,649 (Watanabe et al.) discloses a disposable diaper including a body having a liquid permeable topsheet, a liquid impermeable backsheet, and an absorbent member interposed therebetween. The body is divided into a stomach portion which, when worn, is located on a wearer's stomach side and a back portion which, when worn, is located on his back side. The stomach portion and the back portion are connected and fixed together at both side edge portions, respectively, of the stomach and back portions to form a pair of leg opening portions and a unitary waist opening portion. The waist opening portion and pair of leg opening portions are provided around entire peripheral edges thereof with elastic members forming substantially continuous gathers.

U.S. Pat. No. 5,569,234 (Buell et al.) discloses pull-on garments provided with a continuous belt in the front region and the back region to distribute the forces generated during use in order to better fit the pull-on garment on the wearer. In one embodiment, the pull-on garment comprises a chassis layer comprising a continuous sheet that defines a front region, a back region, and a crotch region; a first belt layer joined to the chassis layer in the front region; a second belt layer joined to the chassis layer in the back region; elastic panel members positioned between the belt layers and the chassis layers which laminate is mechanically stretched to form elastically extensible stretch laminates in both the front region and the back region in the side panels; and seams joining the front region to the back region so as to form the leg openings and waist openings. The continuous chassis layer and belt layers thus form the continuous belt of the pull-on garment. Because the belt layers extend continuously laterally across the waist region and do not cover the entire crotch region, and because the chassis layer is thus exposed in the crotch region, the pull-on garment has a unique aesthetic feature which consumers perceive as providing a cloth-like feel in the waist but the containment characteristics in the crotch

U.S. Pat. No. 6,478,786 (Glaug et al.) discloses a protective underwear having a crotch portion, a waist portion, a front belly portion and a rear back portion. The waist, belly and back portions include plural transversely oriented elastic threads. The underwear includes an absorbent core. Longitudinally oriented elastic threads are located on opposite sides of the core and intersect the transverse elastic threads to enclose the core. The underwear includes a pair of leg openings, each of which has a first arcuate section, a second arcuate section, and an intermediate section. The first arcuate section includes at least one arcuate elastic thread, as does the second arcuate section. Those threads are spaced by a gap. A portion of the longitudinally oriented elastic threads on either side of the core is located closely adjacent a respective gap between the arcuate elastic threads to elasticize the leg openings.

U.S. Pat. No. 5,885,686 (Cederblad et al.) discloses a bicomponent elastomeric extruded netting having bidirectional elasticity, the netting being formed of one set of relatively elastic, extruded strands in one direction and another set of transverse, relatively elastic extruded strands. The strands are formed of relatively elastic resins.

### SUMMARY OF THE INVENTION

The present specification discloses, among other things, one or more novel methods and apparatuses related to absorbent articles such as, for example, diapers, training pants or incontinence articles.

According to one aspect of the teaching disclosed herein, an elasticized retaining panel for an absorbent garment is provided, the panel having a stretch axis defining a stretch direction and a cross axis defining a cross direction generally normal to the stretch axis, the panel comprising an outer web, an inner web overlying the outer web, and an elastic web disposed between the outer and inner webs and bonded thereto. The elastic web includes a plurality of stretch strands extending parallel to the stretch axis and a plurality of support strands extending parallel to the cross axis. The support strands and stretch strands define a plurality of intersection points at which the support strands and the stretch strands are joined together. The stretch strands are elastically extendible in the stretch direction and are extended in the stretch direction relative to the outer and inner webs for exerting an elastically contractile force on the outer and inner webs. The support strands are generally unextended in the cross direction relative to the inner and outer webs.

The support strands and the stretch strands can be constructed of the same material. The support strands can have a support strand thickness and the stretch strands have a stretch strand thickness, and the support strand thickness can be greater than the stretch strand thickness. The elastic web can be bonded to the inner and outer webs with the stretch strands in the extended condition. The panel can include an adhesive layer applied to interior facing surfaces of the inner and outer webs. The adhesive layer can be applied generally continuously over an area coextensive with the panel. The support strands can be spaced apart from each other in the stretch direction by a support strand spacing defined by the spacing between the support strands when the stretch strands are in the relaxed condition, and the stretch strands are spaced apart from each other in the cross direction by a stretch strand spacing, the support strand spacing being greater than the stretch strand spacing. The support strand spacing can be about twice that of the stretch strand spacing. The support strand spacing can be about 4 to 12 mm and the stretch strand spacing can be about 2 to 6 mm.

According to another aspect, an absorbent article having a longitudinal axis defining a longitudinal direction and a transverse axis defining a transverse direction generally normal to the longitudinal axis is provided. The article comprises an absorbent assembly including a core, and a shell for holding the absorbent assembly, the shell including at least one elasticized retaining panel for providing a body-conforming fit about the hips of a user. The elasticized retaining panel includes an outer web, an inner web overlying the outer web, and an elastic web disposed between the outer and inner webs and bonded thereto. The elastic web includes a plurality of stretch strands extending parallel to the transverse axis and a plurality of support strands extending parallel to the longitudinal axis. The support strands and stretch strands define a plurality of intersection points at which the support strands and the stretch strands are joined together. The stretch strands are elastically extendible in the transverse direction and are extended in the transverse direction relative to the outer and inner webs exerting an elastically contractile force on the outer and inner webs. The support strands are generally unextended in the longitudinal direction relative to the inner and outer webs.

The absorbent article can include a front body portion, a rear body portion, and a crotch portion extending therebetween, the front and rear body portions defining ear portions at transversely opposing ends thereof, and wherein each ear portion is provided with a respective retaining panel. Each ear portion can extend transversely outwardly to a respective seam edge, and the respective retaining panel can be spaced transversely inwardly from the seam edge to define a seam margin between the seam edge and the retaining panel, the seam margin being free of the elastomeric web. The respective retaining panels in the front and rear body portions can be spaced apart transversely to define an inelastic central zone underlying the core. The absorbent article can include leg gather elastics extending longitudinally along either side of the core, the leg gather elastics being adhered to the shell between opposed front and rear leg elastic ends. The retaining panels in the ear portions extending from the front body portion can be spaced longitudinally apart from the front leg elastic ends to provide a gap between the front leg elastic ends and a proximate strand of the stretch strands. The retaining panels in the ear portions extending from the rear body portion can extend longitudinally towards rear edges of the leg openings to provide intersection between the rear leg elastic ends and a proximate strand of the stretch strands. The retaining panels in the ear portions extending from the rear body portion can extend longitudinally into the rear leg openings to provide elasticization of a rear edge thereof.

According to another aspect, a belt segment for use in an absorbent article is provided. The belt segment includes an outer web extending in a transverse direction between a left and a right side edge, an inner web extending between the left and right side edges and overlying the outer web, and a pair of elastic retaining panel portions each comprising an elastic web sandwiched between portions of the inner and outer webs and bonded thereto, the retaining panel portions being spaced apart in the transverse direction to define an inelastic central zone between the retaining panel portions. Each elastic web includes a plurality of stretch strands extending in the transverse direction and a plurality of support strands extending in a longitudinal direction, generally normal to the stretch strands, the support strands and stretch strands defining a plurality of intersection points at which the support strands and the stretch strands are joined together. The stretch strands are elastically extendible in the transverse direction and are extended in the transverse direction relative to the outer and inner webs for exerting an elastically contractile force on the outer and inner webs. The support strands are generally unextended in the longitudinal direction relative to the inner and outer webs.

Each retaining panel can be spaced transversely inwardly from the left and right side edges, respectively, to define inelastic seam margins adjacent the left and right side edges. The inner and outer webs can extend longitudinally between upper and lower edges, the upper and lower edges extending transversely between the left and right side edges at respective upper and lower ends thereof, and each retaining panel can be spaced longitudinally inwardly from the upper and lower edges of the inner and outer webs.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a side view in cross-section of an example of an elasticized retaining panel shown in an extended condition;

FIG. 2 shows the retaining panel of FIG. 1 in a contracted condition;

FIG. 3 is an exploded perspective view of the retaining panel of FIG. 1;

FIG. 4 is an enlarged perspective view of an elastic web element of the retaining panel of FIG. 3, shown in the relaxed (contracted) condition;

FIG. 5 is a side view in cross-section of the panel of FIG. 1 showing additional features thereof;

FIG. 6 is a perspective view of an example of an absorbent article;

FIG. 7 is a partial cut-away plan view of the article of FIG. 6;

FIGS. 8-12 are cross-sectional views of the article of FIG. 7, taken across respective section lines 8-8, 9-9, 10-10, 11-11, and 12-12; and

FIG. 13 is an exploded perspective view of a belt segment of the article of FIG. 6.

### DETAILED DESCRIPTION OF THE INVENTION

The following description includes examples of various methods, systems, apparatuses, or processes to illustrate aspects of the teaching disclosed herein. The examples described herein are illustrative and are not intended to limit the scope of any invention that is defined by one or more claims. The examples and embodiments described herein can be modified without departing from the scope of one or more inventions as defined by one or more claims. A single claimed invention can include features from one or more examples or embodiments, and the claimed inventions are not limited to examples or embodiments having all of the features of any one example or embodiment described herein, or having features common to multiple or all of the examples or embodiments described herein. The teaching herein may include particular subject matter that is beyond the scope of the claims appended hereto. The applicants, owners, or inventors retain all rights to such particular subject matter (including, for example, the right to file divisional or continuation applications related thereto) and do not merely by the absence of inclusion in claims appended hereto abandon, disclaim, or dedicate to the public any rights to such particular subject matter.

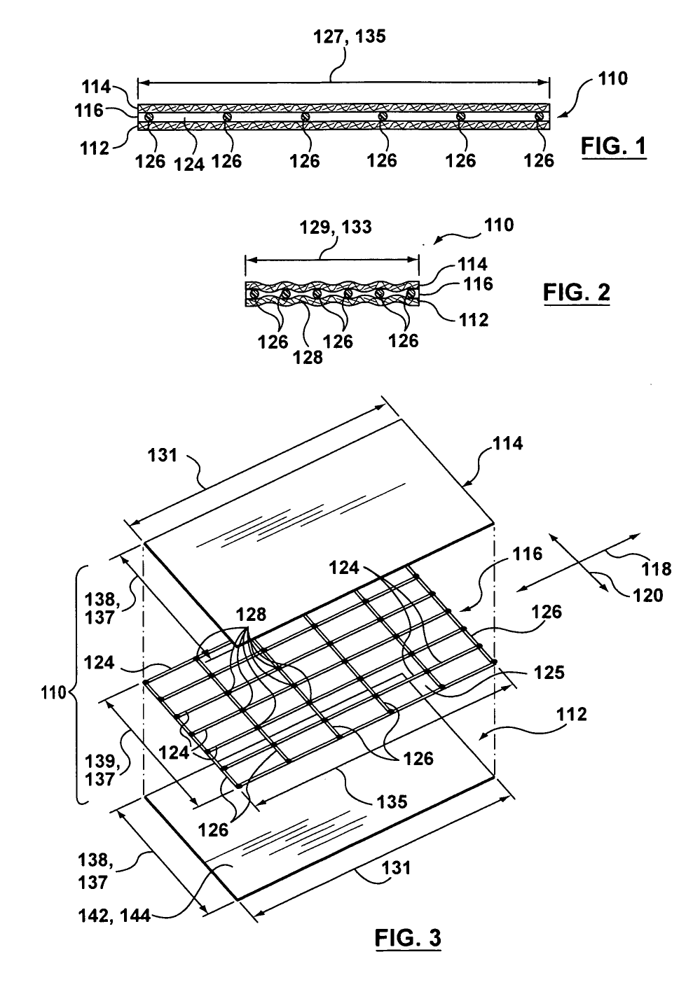

Referring to FIGS. 1-3, an example of an elasticized retaining panel 110 according to teaching disclosed herein is illustrated. The retaining panel 110 includes an outer web 112, an inner web 114 overlying the outer web 112, and an elastic web 116 disposed between the outer and inner webs 112, 114 and bonded thereto.

The retaining panel 110 has a stretch axis 118 extending lengthwise of the panel 110, and a cross axis 120 extending widthwise of the panel 110, normal to the stretch axis 118. Directions parallel to the stretch axis 118 and the cross axis 120 are defined as the stretch direction and cross direction, respectively. The terms “parallel” and “normal” as used herein identify approximate directions and alignments, and are not used in a strict mathematical sense.

The outer and inner webs 112, 114 can be in the form of fabric layers, and/or can be layers comprising natural or synthetic fibers or a combination thereof. The outer and inner webs 112, 114 can be constructed of the same material or can be of distinct materials. In the example illustrated, the outer web 112 is a nonwoven material having a basis weight of about 15 gsm. The inner web 114 is, in the example illustrated, of the same material as the outer web 112.

The elastic web 116 can comprise a network of reticulated strands, including, for example, a plurality of stretch strands 124 extending generally parallel to each other and to the stretch axis 118 and a plurality of support strands 126 extending generally parallel to each other and to the cross axis 120. The stretch strands 124 do not intersect other stretch strands, and the support strands 126 do not intersect other support strands. In the example illustrated, the stretch strands 124 and support strands 126 define a plurality of intersection points 128 at which the stretch strands 124 and support strands 126 cross and are joined together. The intersecting stretch strands 124 and support strands 126 define openings 125 in the elastic web 116 between the strands 124, 126. The openings 125 are, in the example illustrated, of a generally rectangular shape.

The stretch strands 124 are elastically extendible along the stretch axis 118. This means, for example, that a unit length of the stretch strands 124 can be lengthened by applying a tensioning force on the stretch strands 124, and upon release of the tensioning force, the stretch strands 124 will return to (or at least nearly to) the original unit length. The unit length can be measured with the stretch strands 124 in a relaxed state, corresponding to the absence of all or almost all tensioning force on the stretch strands 124.

The stretch strands 124 can be joined to at least one of the outer and inner webs 112, 114 to impart elasticity to the retaining panel 110. In the example illustrated, the panel 110 has an extended panel length 127 (FIG. 1) and a contracted panel length 129 (FIG. 2), the lengths 127, 129 being measured parallel to the stretch axis 118. The outer and inner webs 112, 114 have a neutral length 131 extending along the stretch axis 118, the neutral length 131 defined as the lengths of the webs 112, 114 when taut along the stretch axis 118, free of any contraction (or shortening) by the stretch strands 124. In the example illustrated, the neutral length 131 of the webs 112, 114 is generally equal to the extended length 127 of the panel 110.

The elastic web 116, and the stretch strands 124 thereof, have a relaxed stretch length 133 defined by the length of the stretch strands 124 when in a substantially straight but untensioned condition. In the example illustrated, the relaxed stretch length 133 is generally equal to the contracted panel length 129. The elastic web 116 (and strands 124 thereof) have an extended stretch length 135 that is generally equal to the extended panel length 127. Thus in the illustrated example of the panel 110, the stretch strands 124 are extended relative to the outer and inner webs 112, 114 (in the neutral condition i.e. at neutral length 131, as measured parallel to the stretch strands) and so exert an elastic contractile force on the outer and inner webs 112, 114. When free of any external force, the stretch strands 124 contract the length of the retaining panel 110 along the stretch axis 118 from the extended length 127 to the contracted length 129, forming wrinkles 128 in the outer and inner webs 112, 114.

The panel 110 has a width 137 extending in the cross direction (along the direction that the cross axis 120 extends). The panel width 137 is generally equal to the web width 138 of the webs 112, 114 and the support strand length 139 of the support strands 126.

The support strands 126 can be, but need not be, elastically extendible along their length (i.e. along the direction that the cross axis 120 extends). In the example illustrated, the support strands 126 are made of the same material as the stretch strands 124, and thus would, in the absence of any external constraints, be elastically extendible. Providing support strands 126 and stretch strands 124 of the same material can facilitate manufacture (and reduce cost) of the elastic web 116, and can simplify adhering the strands 124 and 126 to the webs 112, 114 with, for example, a single adhesive.

Notwithstanding the elastic properties thereof, the support strands 126 can be joined to the webs 112, 114 in an unextended condition relative thereto along the cross axis 120. In the embodiment illustrated, the support strand length 139 of the support strands 126 in the panel 110 corresponds to a relaxed (generally straight but untensioned) condition of the support strands 126. The panel 110 in the example illustrated is thus free of elasticity in the direction of the cross axis 120.

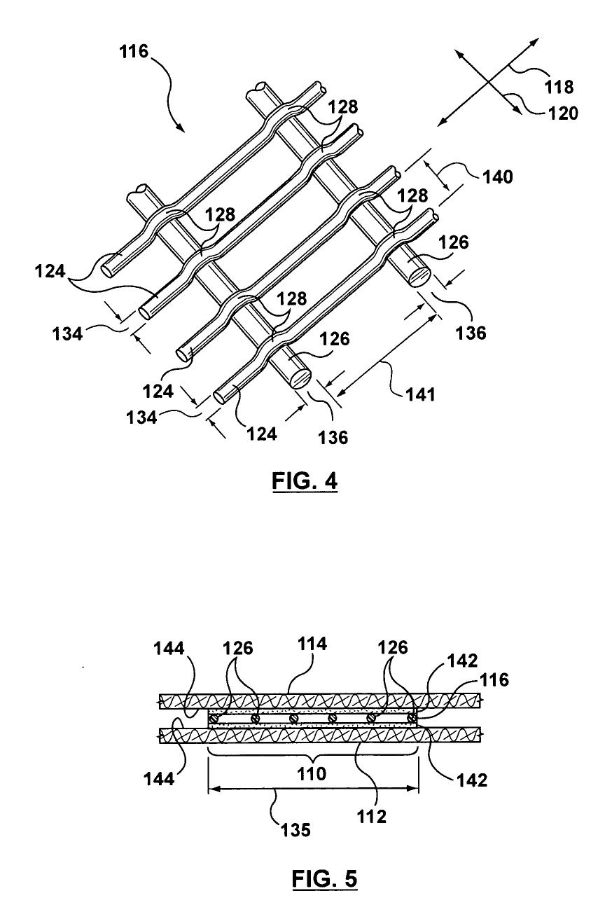

Referring to FIG. 4, in the example illustrated, the stretch strands 124 are constructed of a segmented polyurethane-based material. In the example illustrated, each stretch strand 124 has a thickness 134 (measured parallel to the cross direction) of about 0.23 to 0.26 mm. The elastic layer 116 in the example illustrated has a basis weight of about 45 gsm, and has a maximum extension of about 4 to 5 times the relaxed length in the stretch direction.

The support strands 126 have a thickness 136, measured parallel to the stretch axis 118, that can advantageously be thicker than the stretch strand thickness 134. In the example illustrated, the support strand thickness 136 is about 0.30 to 0.36 mm. The increased thickness 134 of the support strands 126 can help to reinforce the joining of the stretch strands to the webs 116, as described in greater detail subsequently herein.

The support strands 126 are spaced apart from each other in the stretch direction by a support strand spacing 141 defined (in the example illustrated) by the spacing between the support strands 126 when the stretch strands 124 are in the relaxed condition, and the stretch strands 124 are spaced apart from each other in the cross direction by a stretch strand spacing 140. In the example illustrated, the support strand spacing 141 is greater than the stretch strand spacing 140. Accordingly, the relatively higher number of stretch strands 124 can provide the panel 110 with desired tensioning characteristics, and the relatively lower number of support strands 126 can reduce the overall basis weight (i.e. bulk) of the elastic web 116 and hence of the panel 110. The support strand spacing 141 can be about twice that of the stretch strand spacing 140. The support strand spacing 141 can be about 4 to 12 mm and the stretch strand spacing 140 can be about 2 to 6 mm.

As seen in FIG. 5, to bond the elastic web 116 to at least one of the outer or inner webs 112, 114, a layer of adhesive 142 can be applied to the interior surface 144 thereof, and the elastic web 116 can then be sandwiched between the outer and inner webs 112, 114, against the layer of adhesive 142. In the example illustrated, a layer of adhesive 142 is applied to the interior (facing) surfaces 144 of each of the outer and inner webs 112, 114 of the panel 110. The layers of adhesive 142 are provided in a patch having a length and width that match the length 127 and width 137 of the panel 110.

It should be noted that the size or extent of the adhered area of the elastic web 116 defines the extent of the panel 110. In some examples, the adhered area of the elastic web 116 can be less than the surface area of one or both webs 112, 114 between which the elastic web 116 is sandwiched, in which case the panel 110 comprises portions of the webs 112, 114 corresponding in size to the adhered area of the elastic web 116. In some examples, the shape of the adhered area of the elastic web 116 and/or of the adhesive patch may be other than rectangular and the corresponding panel 110 may be other than rectangular in shape.

To provide the stretch strands 124 in an extended condition relative to the webs 112, 114 in the panel 110, the webs 112, 114 can, for example, be held in a taut condition along the stretch axis 118, and the strands 124 can be held in a stretched condition (i.e. to extended length 135) when bonded thereto. Alternatively, the stretch strands 124 can be bonded in a relaxed condition (i.e. length 133) to tautly held webs 112, 114, and the webs 112, 114 can thereafter be mechanically strained in direction of the stretch axis 118 to lengthen the webs (i.e. to provide webs having neutral length 131 that is greater than the length of the webs 112, 114 prior to bonding). Other methods of providing stretch strands 124 in an extended condition relative to neutral length webs 112, 114 may also be employed.

In the example illustrated, the elastic web 116 is stretched or pre-lengthened in the stretch direction 118 prior to bonding between the outer and inner webs 112, 114. The stretched length at the time of bonding can be about 1.5 to 3.5 or more times the relaxed length of the stretch strands 124, and in the example illustrated is about 2 to 2.5 times the relaxed length of the stretch strands 124. The adhesive 142 can be a quick-set adhesive so that the duration of time for which the stretch strands 124 must be held stationary in an extended condition relative to the webs 112, 114 can be minimized.

The construction of the panel 110 as described above can facilitate providing a reliable, consistent bond of the elastic web 116 to the outer and inner webs 112, 114. The stretch strands 124 can, because of their pre-tensioning, tend to try to shorten and pull free from the outer and inner webs 112, 114. The direction of force is along the length of the stretch strands 124, and along the length of (or parallel to) bond lines of adhesive 142 in contact with the stretch strand 124 for bonding the stretch strand 124 to the outer and inner webs 112, 114.

Providing layers of adhesive 142 on the interior surfaces 144 of both webs 112, 114 can help to strengthen the bond of the stretch strands 124 to the webs 112, 114 by providing two parallel bond lines of adhesive, one on either side of each strand 124.

The inventors have discovered that the support strands 126 further facilitate holding the stretch strands 124 in position relative to the outer and inner webs 112, 114. The support strands extend in the cross direction 120, perpendicular to the stretch strands 124, thereby providing transverse bond lines of adhesive that help to anchor the stretch strands 124 to the outer and inner webs 112, 114. In the openings 125 between the stretch strands 124 and support strands 126, the outer and inner webs 112, 114 can bond to each other, forming a transverse bond barrier helping to fix the support strands 126 against movement in the stretch direction 118. By attaching the support strands 126 to the webs 112, 114 in a relaxed state, the support strands 126 will not themselves exert any force trying to free the support strands 126 from the webs 112, 114. Increasing the thickness 136 of the support strands 126 can increase the surface area of the support strand bond lines to further fix the support strands 126, and hence the elastic web 116, in position.

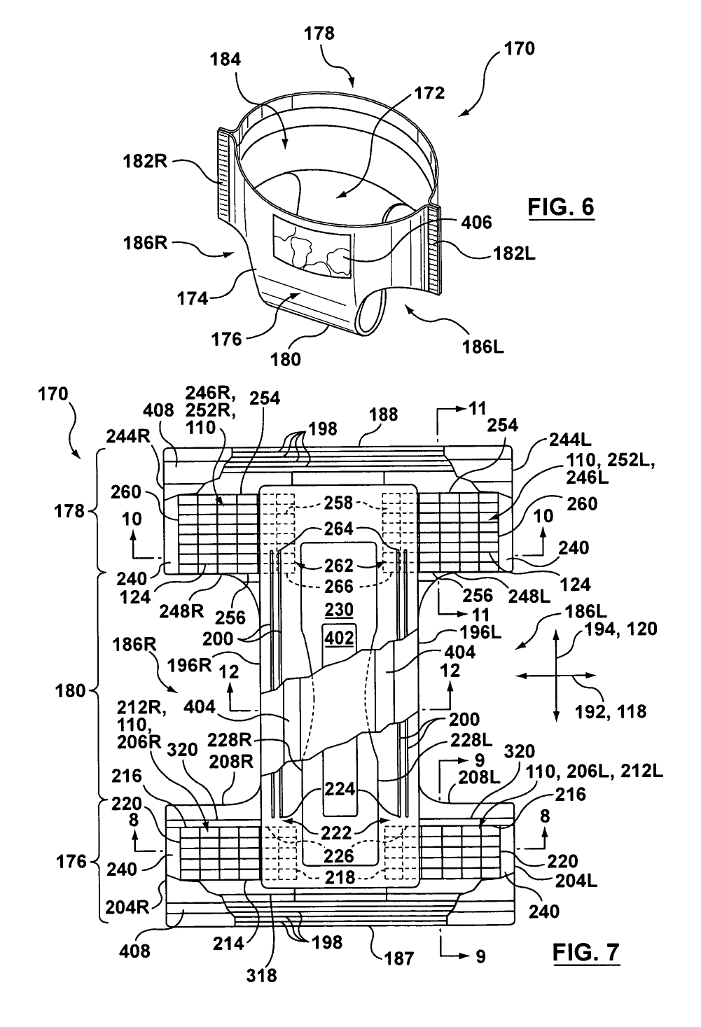

An absorbent article 170 incorporating at least one retaining panel 110 is shown in FIGS. 6 and 7. The article 170 has an absorbent assembly 172 and a shell portion 174 adapted to be secured about the lower torso of a user with the absorbent portion 172 attached thereto and positioned to contact and contain bodily waste discharged by the user.

The article 170 has a front body portion 176, a rear body portion 178, and a crotch portion 180 extending therebetween. The front and rear body portions 176, 178 are joined along respective left and right side seams 182L, 182R to form a waist opening 184 and left and right leg openings 186L, 186R, respectively.

The waist opening 184 is, in the example illustrated, defined by a front waist edge 187 and a rear waist edge 188, each extending in a transverse direction 192 and positioned at opposite ends with respect to a longitudinal direction 194 of the article 170. The leg openings 186 are defined at least in part by left and right central leg opening edges 196L and 196R extending longitudinally along either side of the crotch portion 180.

The waist opening 184 and leg openings 186L, 186R can be elasticized to provide a snug fit about the waist and legs of a user. In the example illustrated, transversely extending waist gather elastics 198 are provided adjacent the front and rear waist edges 187, 188. Longitudinally extending leg gather elastics 200 are provided adjacent the left and right central leg opening edges 196L, 196R.

The article 170 is also provided with at least one retaining panel 110 positioned adjacent the side seams 182L, 182R to provide an elasticized body-conforming fit about the hips of a user. The retaining panel 110 is aligned with the stretch axis 118 thereof (i.e. the stretch strands 124) extending in the transverse direction 192 of the article 170.

It is to be understood that in FIG. 7, the absorbent article 170 is shown in plan view, with the front and rear body portions 176, 178 free of the side seams 182L, 182R and laid open in a flat, unfolded condition. Also, in FIG. 7, the waist and leg gather elastics 198, 200, and the stretch strands 124 of the retaining panel 110, are shown in respective extended positions Also, those features designated by a reference character having a suffix “L” or “R” denote features that are symmetrical about a longitudinal axis that transversely bisects the article 170. Reference herein to those features by reference characters without the “L” or “R” suffix refers generically to such features.

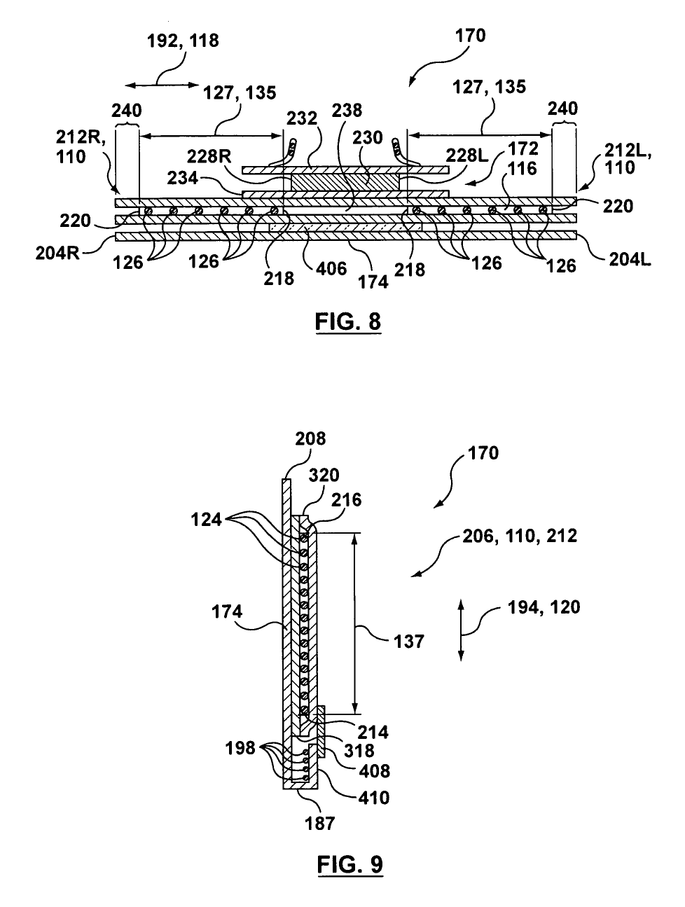

Referring now also to FIG. 8, the front body portion 176 extends transversely between longitudinally extending left and right front seam edges 204L, 204R, respectively, defining a width thereof that is greater than the width of the crotch portion 180. The front body portion 176 is thus provided with left and right front ear portions 206L, 206R, respectively, extending laterally outboard of the transverse extent of the crotch portion 180 (i.e. laterally outboard of the respective left and right central leg opening edges 196L and 196R). The longitudinally inboard extents of the left and right front ear portions 206L, 206R are defined by left and right front leg opening edges 208L, 208R extending transversely between the respective central leg opening edges 196L, 196R and front seam edges 204L, 204R, opposite the front waist edge 187.

In the example illustrated, each of the front ear portions 206L, 206R is provided with a retaining panel 110 to provide an elastically conforming fit about the hips of a user. The stretch strands 124 of each retaining panel 110 in the article 170 are aligned parallel with the transverse axis 192 thereof, and provide elastic extendibility in a circumferential direction with respect to the torso of a user.

In the example illustrated, the retaining panels 110 provided in the left and right front ear portions 206L, 206R are identified as left and right front retaining panels 212L and 212R respectively. The front retaining panels 212L, 212R extend longitudinally between transversely extending upper and lower front panel edges 214, 216, the upper panel edge 214 positioned adjacent the front waist edge 187, and the lower panel edge 216 positioned adjacent the respective left and right front leg opening edges 208L, 208R. The front retaining panels 212L, 212R extend transversely between respective inner side edges 218 proximate the absorbent assembly 172, and outer side edges 220 proximate the front seam edges 204L, 204R. The upper panel edges 214 are spaced inwardly from the front waist edge (towards the leg openings) to provide a waist margin in which the waist elastics can be provided.

The lower front panel edges 216 are, in the example illustrated, generally parallel to and spaced apart from the respective left and right front leg opening edges 208L, 208R. This spacing can facilitate providing a gap 222 between a front-most or forward end 224 of the adhered leg gather elastics 200 and a nearest strand 226 of the stretch strands 124 of the respective retaining panels 212L, 212R. The area of the article 170 spanning across the left and right front leg opening edges 208L, 208R can present a transverse band or line about which the article 170 is subjected to a bending or folding force during use, particularly when the thighs and lower torso of a user are oriented at an acute angle relative to each other, such as when seated. Providing the gap 222 can reduce the bulk of the article 170 and improve the flexibility or pliability of the article 170 along such fold or bend line.

The inner side edges 218 of the front retaining panels 212L, 212R are disposed proximate the absorbent assembly 172. To describe this in greater detail, the absorbent assembly 172 can include a liquid permeable topsheet 232, a liquid impermeable backsheet 234, and an absorbent core 230 sandwiched between the topsheet 232 and backsheet 234. The absorbent core can include a tissue-encased pad of fluff and super absorbent polymer, and a transfer layer disposed between the pad and the topsheet. The absorbent core 230 can extend transversely between left and right core side edges 228L, 228R.

The inner side edges 218 of the front retaining panels 212L, 212R can advantageously be positioned laterally outboard of the core side edges 228L, 228R thereby providing an inelastic central zone 238 between the left and right front retaining panels 212L, 212R. The inelastic central zone 238 underlies a forward-most portion of the core 230, and the absence of elastic strands crossing over part or all of the core can prevent undue stretching forces being applied to the core 230, thus avoiding possible rupture thereof.

The outer side edges 220 of the front retaining panels 212L, 212R can be positioned laterally inboard (or transversely inwardly) of the left and right front seam edges 204L, 204R of the front body portion 176. This spacing can provide left and right side seam margins 240 extending between the outer panel edges 220 of each of the left and right panels 212L, 212R and the respective seam edges 204L, 204R.

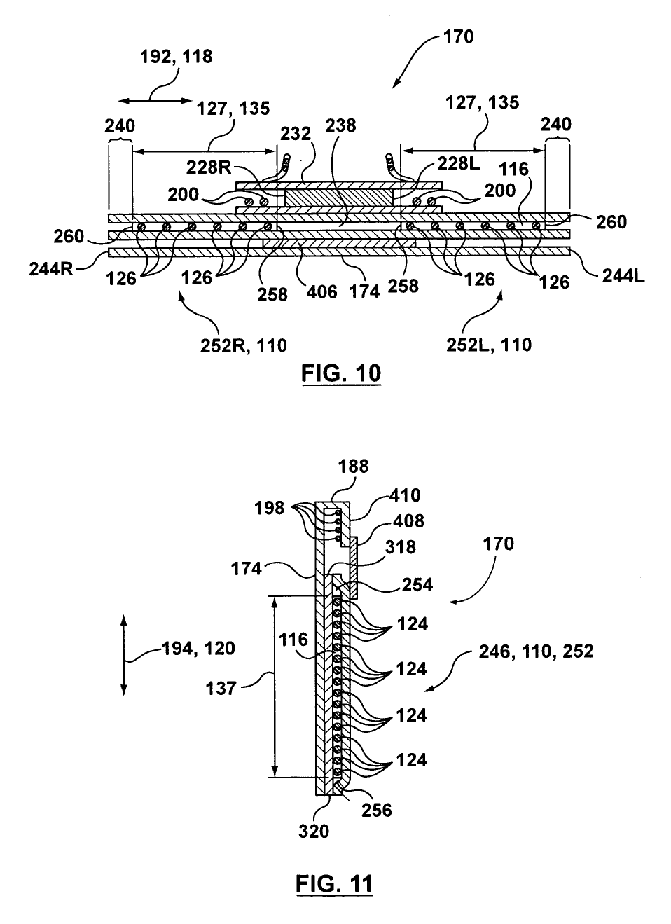

Referring also to FIGS. 10 and 11, the rear body portion 178 extends transversely between longitudinally extending left and right rear side seam edges 244L, 244R, and has left and right rear ear portions 246L, 246R at transversely opposing ends thereof. Left and right rear leg opening edges 248L, 248R extend between the respective central leg opening edges 196L, 196R and rear side seam edges 244L, 244R, defining the longitudinally inboard extents of the left and right rear ear portions 246L, 246R.

Each of the rear ear portions 246L, 246R is, in the example illustrated, also provided with respective retaining panels 110, identified as left and right rear retaining panels 252L, 252R. The rear retaining panels 252L, 252R extend between upper and lower rear panel edges 254, 256, and between inner and outer rear panel side edges 258 and 260, respectively.

Similar to the front panels 212L, 212R, the inner rear panel side edges 258 are spaced laterally outboard of the core side edges 228L, 228R over the transverse length thereof, to provide an inelastic central zone 238 between the panels 252L, 252R. The outer side edges 260 are spaced transversely inwardly of the rear side seam edges 244L, 244R to provide seam margins 240 therebetween.

In the example illustrated, the lower rear panel edge 256 is not spaced apart longitudinally from the rear leg opening edges 248L, 248R, but instead, at least a portion of each rear panel edge 256 is coterminous with a portion of the rear leg opening edges 248L, 248R. This extension of the panels 252L, 252R into the leg openings can help to further elasticize the leg openings, and can provide one or more intersection points 262 (FIG. 7) between rearward ends 264 of the leg gather elastics 200 and one or more proximate strands 266 of the stretch strands 124 of the panels 252L, 252R.

Referring now to FIG. 13, to facilitate providing the panels 110 in the article 170, belt segments 310 can be provided for attachment to the front and rear body portions 176, 178. Each belt segment 310 includes an outer belt web 312 and an inner belt web 314, each belt web extending transversely between longitudinally directed left and right belt side edges 316L, 316R, and extending longitudinally between transversely directed upper and lower belt edges 318, 320. The belt side edges 316L, 316R are, in the example illustrated, positioned for registration with the side seam edges 204, 244 of the front and/or rear body portions 176, 178 of the article 170.

Each belt segment 310 further includes a pair of retaining panels 110 provided therein, and identified as left and right belt retaining panels 322L, 322R. The belt retaining panels 322 are, in the example illustrated, formed in the belt segment 310 by providing a pair of elastic web members 116 sandwiched between transversely spaced apart portions of the outer and inner belt webs 312, 314, and bonded thereto with respective layers of adhesive 142. More particularly, in the example illustrated, left and right patches 324L and 324R of adhesive 142 are provided on interior facing surfaces 144 of the outer and inner belt layers 312, 314. Thus a total of four adhesive patches 324 are provided, one above and below each elastic web 116.

The surface area of the adhesive patches 324 can be sized to generally define the adhered area of elastic web 116, and hence the area of the belt retaining panels 322. In the example illustrated, the belt retaining panels 322 extend transversely (along axis 192, 118) between inner side edges 338 and outer side edges 340. The belt retaining panels 322 extend longitudinally between upper and lower belt panel edges 334 and 336, respectively.

In the example illustrated, the two belt retaining panels 322L, 322R at either side of the belt segment 310 provide a transverse spacing between the inner edges 338 of the belt retaining panels 322L, 322R that corresponds to the inelastic central zone 238 of the article 170.

Each belt retaining panel 322 can be spaced transversely inwardly from the left and right belt side edges 316L, 316R, respectively, to provide a space between the outer side edges 340 of the belt retaining panel 322 and the respective belt side edges 316 of the belt segments 310, the space corresponding to the seam margins 240 adjacent the left and right belt side edges 316L, 316R. The seam margins 240 are adapted to provide sites for ultrasonic bonding. Providing seam margins that are free of adhesive and elastic elements can facilitate ultrasonic bonding through the belt segments 310 at the seam margins 240. Extending the belt segment 310 into the side seams 182L, 182R can facilitate fixing the belt segment 310, and hence the belt retaining panels 322, in position relative to the article 170.

Each retaining panel 322 can be spaced longitudinally inwardly from the upper and lower edges 318, 320 of the outer and inner belt webs 312, 314. This spacing can provide upper and lower belt margins extending longitudinally beyond the corresponding respective upper and lower edges of the retaining panels 110. The upper and lower belt margins can help to ensure that the strands 124, 126 of the elastic web 116 remain encased between the outer and inner belt webs 312, 314, thus avoiding contact of the strands 124, 126 with a user's skin.

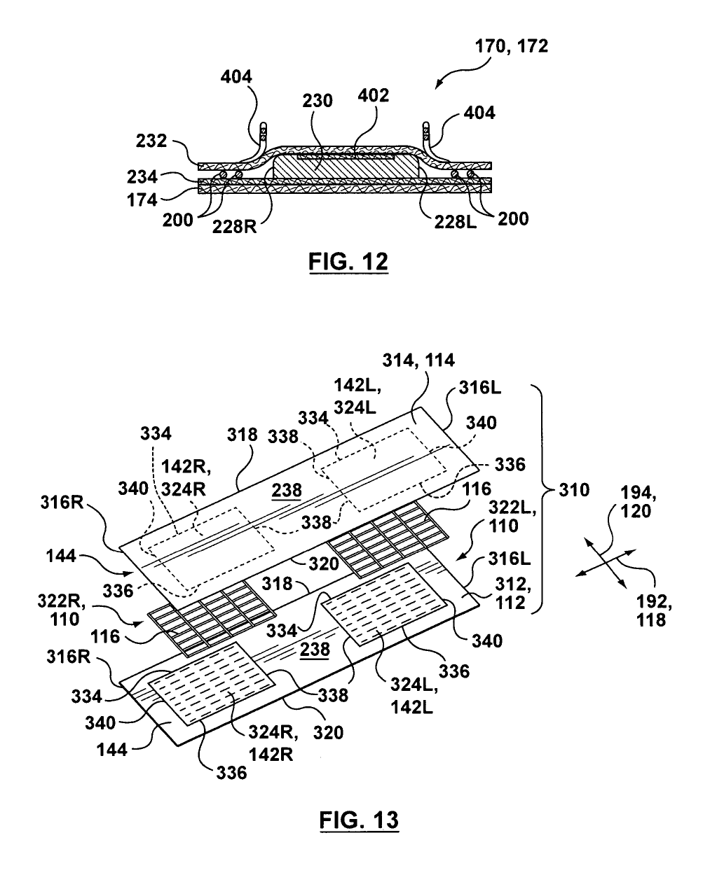

The article 170 can include various additional features to enhance the product. For example, as seen in FIG. 12, the absorbent assembly 172 can include a transfer layer 402 disposed between the core 230 and the topsheet 232. A pair of stand-up cuff elements 404 can be provided on the topsheet 232, one adjacent either side 228L, 228R of the core 230. As seen in FIGS. 6 and 8, the front body portion 176 can be provided with a decorative appliqué or character strip 406 presenting a graphical image visible through the outer layer of the shell 174. The rear body portion 178 can also be provided with a character strip 406 (FIG. 10).

As best seen in FIGS. 6, 7, 9, and 11, the waist opening 184 can be lined with a waist cap 408 that can be in the form of a strip or band of material extending transversely between the side edges 204L, 204R and edges 244L, 244R in each of the front and rear body portions, 176, 178, respectively. The waist cap 408 can extend longitudinally over any seams between a waist fold-over 410 of the shell 174, and upper ends 318 of any adjacent retaining panels 212, 252.

It is to be understood that what has been described are preferred examples of the teaching disclosed herein. The disclosure nonetheless is susceptible to certain changes and formation of alternative examples that remain within the scope of what is taught herein.

We claim:

1. An elasticized retaining panel for an absorbent garment, the panel having a stretch axis defining a stretch direction and a cross axis defining a cross direction normal to the stretch direction, the panel comprising:
a) an outer web;
b) an inner web overlying the outer web; and
c) an elastic web disposed between the outer and inner webs and bonded thereto;
d) the elastic web including a plurality of stretch strands extending generally parallel to the stretch axis and a plurality of support strands extending generally parallel to the cross axis, the support strands and stretch strands defining a plurality of intersection points at which the support strands and the stretch strands are joined together;
e) the stretch strands being elastically extendible in the stretch direction and being extended in the stretch direction relative to the outer and inner webs for exerting an elastically contractile force on the outer and inner webs;
f) the support strands being generally unextended relative to the inner and outer webs in the cross direction.

2. The panel of claim 1 wherein the support strands and the stretch strands are constructed of the same material.

3. The panel of claim 1 wherein the support strands have a support strand thickness and the stretch strands have a stretch strand thickness, the support strand thickness being greater than the stretch strand thickness.

4. The panel of claim 1 wherein the elastic web is bonded to the inner and outer webs with the stretch strands in the extended condition.

5. The panel of claim 1 further comprising an adhesive layer applied to interior facing surfaces of the inner and outer webs.

6. The panel of claim 5 wherein the adhesive layer is applied generally continuously over an area coextensive with the panel.

7. The panel of claim 1 wherein the support strands are spaced apart from each other in the stretch direction by a support strand spacing defined by the spacing between the support strands when the stretch strands are in the relaxed condition, and the stretch strands are spaced apart from each other in the cross direction by a stretch strand spacing, the support strand spacing being greater than the stretch strand spacing.

8. The panel of claim 7 wherein the support strand spacing is about twice that of the stretch strand spacing.

9. The panel of claim 7 wherein the support strand spacing is about 4 to 12 mm.

10. The panel of claim 9 wherein the stretch strand spacing is about 2 to 6 mm.

11. An absorbent article having a longitudinal axis defining a longitudinal direction and a transverse axis normal to the longitudinal axis and defining a transverse direction, the article comprising:
a) an absorbent assembly including a core;
b) a shell for holding the absorbent assembly, the shell including at least one elasticized retaining panel for providing a body-conforming fit about the hips of a user;
c) the elasticized retaining panel including an outer web, an inner web overlying the outer web, and an elastic web disposed between the outer and inner webs and bonded thereto;
d) the elastic web including a plurality of stretch strands extending parallel to the transverse axis and a plurality of support strands extending parallel to the longitudinal axis, the support strands and stretch strands defining a plurality of intersection points at which the support strands and the stretch strands are joined together;
e) the stretch strands being elastically extendible in the transverse direction and being extended in the transverse direction relative to the outer and inner webs for exerting an elastically contractile force on the outer and inner webs;
f) the support strands being generally unextended relative to the inner and outer webs in the longitudinal direction.

12. The absorbent article of claim 11, further comprising a front body portion, a rear body portion, and a crotch portion extending therebetween, the front and rear body portions defining front and rear ear portions at transversely opposing ends thereof, and wherein each ear portion is provided with a respective retaining panel.

13. The absorbent article of claim 12, wherein each ear extends transversely outwardly to a respective seam edge, and wherein the respective retaining panel is spaced transversely inwardly from the seam edge to define a seam margin between the seam edge and the retaining panel, the seam margin being free of the elastomeric web.

14. The absorbent article of claim 13 wherein the respective retaining panels in the front and rear body portions are spaced apart transversely to define an inelastic central zone underlying the core.

15. The absorbent article of claim 12 further comprising leg gather elastics extending longitudinally along either side of the core, the leg gather elastics being adhered to the shell between opposed front and rear leg elastic ends.

16. The absorbent article of claim 15 wherein the retaining panels in the front ear portions are spaced longitudinally away from the front elastic ends to provide a gap between the front leg elastic ends and a proximate strand of the stretch strands.

17. The absorbent article of claim 15 wherein the retaining panels in the rear ear portions extend longitudinally towards rear edges of the leg openings to provide intersection between the rear leg elastic ends and a proximate strand of the stretch strands.

18. The absorbent article of claim 15 wherein retaining panels in the rear ear portions extend into the rear leg openings to provide elasticization of a rear edge thereof.

19. A belt segment for use in an absorbent article, comprising:
a) an outer web extending in a transverse direction between a left and a right side edge;
b) an inner web extending between the left and right side edges and overlying the outer web; and
c) a pair of elastic retaining panel portions each comprising an elastic web sandwiched between portions of the inner and outer webs and bonded thereto, the retaining panel portions being spaced apart in the transverse direction to define an inelastic central zone between the retaining panel portions;
d) each elastic web including a plurality of stretch strands extending in the transverse direction and a plurality of support strands extending in a longitudinal direction, the longitudinal direction being generally normal to the transverse direction, the support strands and stretch strands defining a plurality of intersection points at which the support strands and the stretch strands are joined together;
e) the stretch strands being elastically extendible in the transverse direction and being extended in the transverse direction relative to the outer and inner webs for exerting an elastically contractile force on the outer and inner webs; and
f) the support strands being generally unextended in the longitudinal direction relative to the inner and outer webs.

20. The belt segment of claim 19 wherein each retaining panel is spaced transversely inwardly from the left and right side edges, respectively, to define inelastic seam margins adjacent the left and right side edges.
